(19) United States
(12) Patent Application Publication
(10) Pub. No.: US 2005/0074422 A1
(43) Pub. Date: Apr. 7, 2005

(54) Fixative

(75) Inventor: Francesco Visinoni, Mozzo (BG) (IT)

Correspondence Address: FITZPATRICK CELLA HARPER & SCINTO, 30 ROCKEFELLER PLAZA, NEW YORK, NY, 10112, US

(73) Assignee: Milestone S.r.l., Sorisole (BG) (IT)

(21) Appl. No.: 10/619,443

(22) Filed: Jul. 16, 2003

(30) Foreign Application Priority Data
Mar. 5, 2003 (EP) 03 005 010.8

(51) Int. Cl. A61K 7/06; A61K 7/11

(52) U.S. Cl. 424/70.13

(57) ABSTRACT

The present invention describes a fixative composition comprising ethanol, water, 1,2-propanediol, polyvinyl alcohol and an effective amount of at least one monomeric polyhydroxy compound. The fixative composition may be used in histopathology, cytology and immunohistochemistry.

The present invention is directed to an ethanol-based fixative, a kit comprising the fixative and their use in histopathology, cytology and immunohistochemistry.

In surgical pathology practice the most widely used fixative is an aqueous solution of formaldehyde which is called formalin and used at a concentration of 4%. The first mention of formaldehyde as a fixative was by F. Blum in 1896 (source: The Journal of Histotechnology, Vol. 24, No. 3, September 2001, pages 155 to 162).

After fixation, all the specimens are dehydrated in an increasing concentration of ethanol, put in xylene for clearing before impregnation in paraffin to obtain blocks. Those blocks will be cut in tiny slices to a thickness of some microns and stained with the standard hematoxylin-eosin for morphological evaluation.

The time required for the traditional fixation and processing of a specimens is usually between 24 and 48 hours. In case of biopsy materials which are commonly rather small fragments, it may be reduced to some hours.

In addition, in the last years, the use of microwaves has dramatically reduced the fixation and processing periods resulting in obtaining so-called “same day diagnosis”, even with surgical specimens.

It is known that the use of aldehydes for fixation has some severe disadvantages. Formalin is irritating for the mucosae and has also been indicated as a carcinogen for oropharynx and respiratory tract. For these reasons, it can be only used with caution requiring safety standards such as working under the hood, wearing protective gloves and goggles, with specific measurements concerning formalin contaminated waste (The Journal of Histotechnology, Vol. 24, No. 3, September 2001, pages 165 to 175).

It is also known that formalin causes important protein-protein and nucleic acid—protein cross linkings (The Journal of Histotechnology, Vol. 24, No. 3, September 2001, pages 151 and 152). This makes it difficult to extract undamaged mRNA and/or DNA from formalin fixed paraffin processed tissues. Therefore, recovery of intact mRNA and/or DNA molecules from tissue samples which have been fixed with formalin and processed in paraffin is under question. With the increasing importance of molecular biology studies and methods, needing the recovery of intact mRNA and/or DNA molecules, these cross linking effects have become a severe limitation concerning the use of such material for molecular biology studies, in particular for gene-profiling analysis.

The need of material suitable for molecular analysis has become very important in the last years, especially after the completion of the Human Genome Project, and these studies will give important insights in the nature of many diseases, with important therapeutical implications. Despite the efforts to obtain optimal genetic material from formalin-fixed specimens, the results are substantially poor and not uniform.

To prevent nucleic acids degradation, fresh material is usually taken, frozen in liquid nitrogen immediately after surgical operation and kept in special refrigerators at −80° C.

For this reason part of the specimen is no more suitable for morphological analysis, but if still a diagnosis has to be made with frozen sections, many artefacts due to freezing will occur. Another possibility is to fix the frozen material and process it routinely, but also this material will show many artifacts and its antigenicity may be compromised, with possible errors in the interpretation of the results of immunoreactions. Finally, the genetic material obtained from fresh specimens is derived from all the cells present, not only from the cells to be investigated.

For these reasons, there has been an increasing interest concerning alternative fixatives which allow a complete morphological evaluation of the material together with recovery of good quality DNA/RNA/proteins. Paraffin blocks are much more easy to handle and store than frozen material. In addition, the researchers will be enabled to collect particular areas of the specimen using Laser Capture Microdissection, to study tumor heterogeneity, without contamination from other cells present in the background substance (connective cells, lymphocytes, etc).

The formalin alternatives can be subdivided in two broad categories:
Alcohol-based
non alcohol-based

The first group comprises ethanol as such, some well-known mixtures (for example Carnoy's fixative and its derivative Methacarn, both containing chloroform and the latter methanol instead of ethanol) and some other fixatives such as NeoFix and Kryofix).

The second group includes fixatives which are almost all based on glyoxal, a dialdehyde less hazardous than formalin, however, having a similar action on cellular components.

At present, there are only few works concerning the use of the above alternatives for molecular studies, but the results clearly indicate that ethanol and ethanol-based fixatives give at least better results than formalin (source: “Evaluation of Non-Formalin Tissue Fixation for Molecular Profiling Studies” by John W. Gillespie et al. published in the American Journal of Pathology, Vol. 160, No. 2, February 2002.

Ethanol acts as a fixative causing protein denaturation, with little or no degradation of the nucleic acids. There are, however, some limitations for molecular studies: the best results are obtained with a low temperature fixation (4° C.) and with a low temperature polyester resin embedding (at 38° C.). At present there is only one work on this application, performed on prostatic tissue. The use of pure ethanol for fixation usually causes a significative shrinkage at the edges of the specimens, together with some nuclear artifacts. When ethanol is used for small biopsies, the shrinkage of the tissue can affect a correct diagnosis.

Recent developments in this field have revealed that while alcoholic mixtures used as a fixative can lead to some acceptable results, optimum results have never been obtained. A variety of ethanol-based fixative is described in the Journal of Histotechnology, Vol. 23, No. 4, December 2000, pages 299 to 307.

It is therefore object of the invention, to provide a fixative as a formalin substitute which allows a rapid fixation, gives accurate morphological details and exhibits a good recovery of nucleic acids and proteins.

This object has been solved by the fixative composition of the present invention.

The present invention is defined in claim 1 of the present application.

Claim 1 concerns a fixative composition which comprises, as essential components, ethanol, water, 1,2-propanediol, polyvinyl alcohol and an effective amount of at least one monomeric polyhydroxy compound.

The sub-claims define preferred embodiments of the fixative composition of the present invention.

In addition, the present invention concerns a kit for fixation of tissue comprising a first means of keeping a mixture of water, 1,2-propanediol, polyvinyl alcohol and an effective amount of at least one monomeric polyhydroxy compound and a second means for keeping ethanol.

The sub-claims define preferred embodiments of the kit of the invention. The fixative composition and the kit of the present invention are perfectly suited for investigation of histological and cytological specimens. The fixative composition as well as the kit are of an extremely low toxicity due to their ethanol content and they have been found to be absolutely compatible with all histoprocessors such as traditional and microwave stimulated ones.

It has been shown that the introduction of an effective amount of a monomeric polyhydroxy compound in a mixture of ethanol, water, 1,2-propanediol and polyvinyl alcohol result in the provision of a highly effective fixative for histological and cytological as well as immunohistochemical purposes. The fixative of the invention is optimized for a rapid microwave-stimulated fixation and processing giving good morphological details and optimal expression of antigenic properties of various tissues and finally has demonstrated very good preservation of nucleic acids and proteins.

It has been revealed that the best result can be obtained when using a carbohydrate comprising at least three carbon atoms as the monomeric polyhydroxy compound. Experimentally best results have been obtained by using carbohydrates with 6 carbon atoms. Examples for this are hexitols such as sorbitol, mannitol and dulcitol, D-sorbitol being preferred.

The monomeric polyhydroxy compound is preferably contained in the fixative in an amount of 0.05 to 2.00 wt.-%.

Ethanol is the main component of the fixative composition of the present invention. It should be contained in an amount of 50 to 85 wt.-%, 65 to 75 wt.-% being preferred.

As already mentioned in the beginning, ethanol has already been used in pathology, commonly at a concentration of 70%. In addition, it has a powerful germicide action.

The function of ethanol is to denature proteins by its dehydrating action, with a disruption of the tertiary structure thereof. This action is at least partially reversible by protein renaturation.

In the fixative composition of the present invention, it has also been chosen for its low toxicity towards methanol. Furthermore, it has been found not to interfere (as isopropylic alcohol does) with some histochemical stains.

In addition, denaturated ethanol does render the fixative very cheap. The final concentration in the present fixative is preferably 72%.

Water is contained in the fixative composition of the present invention to allow the dissolution of water soluble components in the ethanol. Water is cheap and absolutely harmless. The amount of water in the fixative composition of the present invention ranges preferably from 10 to 40 wt.-%.

1,2-propanediol is contained as a further essential component because of its anti-freezing properties. It lowers the freezing point of water and exhibits mutual solvent properties. It is perfectly soluble in water and in lipids under formation of oil-in-water emulsions and it is well-known plasticizer. It is non-toxic and widely used in cosmetics, food products and pharmaceutical formulations. Its relatively low molecular weight allows a rapid penetration in tissues and cells and its anti-freezing properties protect the tissue against the effects of low temperatures. It has been shown that 1,2-propanediol is preferably present in the fixative composition of the present invention in an amount of 3 to 20 wt.-%.

Polyvinyl alcohol is commonly known as a tissue protectant in enzyme histochemistry contributing to a reduction of the artefacts produced by freezing, cutting and thawing. The function of polyvinyl alcohol is to trap the water molecules, thus leaving less solvent water available for diffusion of molecules from the tissue section. For this reason many cellular components are “blocked” at their site inside the cell and thus can be more easily localized. Another advantage in cytological specimens is the prevention of cells' loss during staining procedures. This action may play a role in preventing the detachment of cells from amorphous material which is sometimes observed in surgical specimens (e.g. necrotic material, mucus, etc).

The monomeric polyhydroxy compound contained in the fixative composition of the present invention, at least those comprising at least three carbon atoms are widely used in industry as an oil absorber, an organic solvent and as a sweetener (sugar substitute).

The low molecular weight of for example sorbitol, mannitol and dulcitol and the water solubility thereof allow rapid penetration in tissues and cells. The main function of said polyhydroxy compound is to prevent the adverse effects of ethanol, particularly the plasticizing effects thereof. They have been found to act as a thermal stabiliser and anti-denaturant for proteins and they have been evidenced to exhibit a certain kryoprotectant activity.

It has been surprisingly shown that the specific combination of components constituting the fixative composition of the present invention gives raise to a perfectly working fixative which allows a complete morphological evaluation of the tissue material together with a recovery of intact proteins and nucleic acids. Even though the working mechanism of the combined fixative is not bound to any theory, it is assumed that the action of ethanol is influenced by the other active components of the mixture, so that at least part of the tissue water is prevented from a escaping giving a less “shrinked” appearance to the cell and the background intercellular substance.

The fixative composition according to the invention may also contain additives and/or auxiliary agents to further improve the efficiency of the fixative.

Examples of the additives and/or auxiliary agents to be added are selected from acceptable salts, alcohols, ketons, carboxylic acids, sugars, polymers, aglycons and polyphenols.

Suitable salts include calcium carbonate (a phospholipid chelating agent), calcium acetate (a phospholipid chelating agent), EDTA, stagnus chloride (for membrane preservation), Na₂HPO₄ (for membrane preservation), MgCl₂, NaCl and zinc sulphate.

The alcohols which may be added to the fixative composition include mono-, di- and trihydric alcohols. Examples of monohydric alcohols are butanol and long chain fatty alcohols such as octanol and decanol. The dihydric alcohols comprise glycols such as ethylene glycol, polyethylene glycol having different molecular weights, pentylene glycol and hexylene glycol.

Examples of trihydric alcohols are triols and polytriols such as glycerol and polyglycerol.

In same cases it can be of value to add polyols such as xylitol and maltitol. The alcoholic additive should be different from the polyhydroxy compound contained in the fixative composition of the invention.

If appropriate, also carboxylic acids may be added such as acetic acid (coagulant of nucleic acids) and carboxylic acids having 5 to 8 carbons atoms.

The sugars include for example fructose, sucrose, trehalose and polysaccarides including locust bean gum, xantahn gum, aratic gum, carboxymethyl cellulose and pectin, as well as carbomer (semisynthetic polysaccharide).

For certain tissue samples the addition of polymers is desirable comprising polyvinyl pyrrolidone, dextran, polyphenols and aglycons.

The amount of the additives and/or auxiliary agents to be used in the fixative composition of the present invention is not critical, however, their content should not exceed 10 g to ensure that the effectiveness of the inventive fixative composition is not affected.

The fixative composition of the present invention may be also provided as a kit for fixation. The kit may comprise a first means for keeping a mixture of water, 1,2-propanediol, polyvinyl alcohol and an effective amount of at least one monomeric polyhydroxy compound and a second means for a keeping ethanol. The contents of the first and second means can be mixed just prior to use to provide a fixative composition. Shelf life of this fixative in kit form ranges from 6 to 9 months. If necessary, the content of the first means can be shipped separately and the user can add the ethanol by himself to prepare a fresh fixative composition.

The means for keeping the components can be any means suitable for storing and shipping. They can be made of plastic or glass material in different sizes and shapes.

In a preferred embodiment of the kit, the monomeric polyhydroxy compound is a carbohydrate comprising at least six carbon atoms. More preferably, said carbohydrate is a hexitol such as sorbitol, mannitol and dulcitol, D-sorbitol being most preferred.

When the contents of the first and the second means are mixed, a fixative is preferably provided comprising 50 to 85 wt.-% of ethanol, 10 to 40 wt.-% of water, 3 to 20 wt.-% of 1,2-propanediol, 0.05 to 3.00 wt.-% of polyvinyl alcohol and 0.05 to 2.00 wt.-% of the at least one monomeric polyhydroxy compound.

In a preferred embodiment of the kit, the mixture of the first means further contains additives and/or auxiliary agents.

The auxiliary agents have the function to improve the efficiency of the fixative. The choice of the appropriate additive(s) and/or auxiliary agent(s) is dependent on the tissue to be examined.

The additives and/or auxiliary agents to be added to the mixture contained in the first means are selected from acceptable salts, alcohols, ketons, carboxylic acids, sugars and polymers. In this context, it is referred to the above-mentioned listing of additives and/or auxiliary agents.

The fixative composition of the present invention as well as the kit according to the present invention may be used for examining tissue samples in histopathology, cytology and immunohistochemistry. The tissue samples to be examined can be any material of human or animal origin.

It has been shown that the inventive fixative composition can be successfully used to test any material excised from the human or animal body. Examples include central nervous system, thyroid, adrenal gland, hypophysis, pancreas, lung and bronchus, heart, gastrointestinal tract (esophagus, stomach, small intestine, large intestine), liver, kidney, bladder, testis, ovary, ulterus, prostate, breast, soft tissues, bone, bone marrow, lymph nodes, spleen.

The material was examined after immersion fixation of the entire organ or part of it, some reduced to small fragments, to simulate a biopsy, others as a specimen normally examined in routine histopathology. Common dimensions of specimens are 25×20 mm with a thickness of 3 to 4 millimetres.

Part of the material has also been fixed in formalin, with the same procedures to obtain “mirror blocks” used for routine stains and for comparisons.

The fixation may be accelerated by use of microwave applying common processing procedures without changes in time schedules or reducing them, or in a microwave processor, with periods varying from 20 minutes for biopsy material to 2 to 3 hours for larger specimens. Using the fixative composition of the present invention, it is possible to reduce the normal time processing from about 16 hours to 4 to 5 hours, for large and fatty specimens (for example breast) simply reducing the thickness thereof to about 2 millimetres. This reduction is due the properties of the fixative, which may be considered also a sort of processing fluid.

Independent of the way of further processing, the samples processed are finally embedded in paraffin blocks.

Moreover, the fixative composition and the kit of the present invention may be used in cytological preparation of several organs (CNS, breast, thyroid, etc.) either by immersing the slides in the fixative or using it as a spray, with optimal preservation of the cytological details.

Some histochemical stains have been performed in various specimens treated with the fixative composition of the present invention: PAS strain, Grocott silver methanamine; Masson's thricrome, giemsa, reticulin, toluidine blue, alcian blue, alcian blue-pas, orcein, PAS-Orange and Ziehl-Neelsen.

There was no variation observed in the staining properties between materials fixed in the fixative composition according to the present invention and formalin-fixed material.

Many specimens (biopsies and surgical specimens) have also been tested for immunohistochemical reactions with optimal results.

In some occasions the antigen retrieval procedures are not necessary while some cases must be slightly changed, for example, by reducing the temperature or changing the buffers. The results have been compared with their expression on formalin fixed material.

Examples of antigens to be tested: antigen of epithelial expression (cytokeratins, epithelial membrane antigen), CD antigens (CD3, 4, 8, 15, 20, 30, 45, 45RO), proliferation markers (Ki67, MIB1), intermediate filaments (vimentin, desmin, GFAP, neurofilaments), hypophyseal markers (prolactin, ACTH, GH, FSH, LH, TSH), estrogen and progesteron receptors and other such as S100 protein, actin, c-erb2, chromogranin, synaptophysin, gastrin, prostatic specific antigen.

Molecular biopsy studies have been performed and the results have been always compared with the same material fixed in formalin. The results clearly indicate a better nucleic acid recovery from tissue materials fixed in the fixative composition of the present invention. The test were performed on: spleen, tonsil, CNS tumor (oligodendroglioma), thyroid, liver (hepatocellular carcinoma) and lung. The formalin fixed material showed degraded DNA, whereas the material fixed in the fixative composition of the present invention showed large quantities of intact DNA, especially of high molecular weight.

The fixative composition of the present invention and the kit of the invention exhibit superior properties over the commonly used fixatives such as formalin and ethanol. The fixative composition and the kit of the invention are advantageous in the following aspects: a simultaneous fixation, dehydration and lipid extracting properties; no shrinkage of tissue; an optimal preservation of morphological details; an extremely low toxicity; an optimal preservation of tissues' antigenic properties with reduction of the use of antigen retrieval procedures; optimal staining properties (hematoxylin and eosin and histochemical stains); an optimal preservation of nucleic acids for molecular studies; suitable as a fixative for cytological specimens and an optimal preservation of the morphology after a prolonged period of tissue's freezing.

1. A fixative composition comprising:
(i)—ethanol;
(ii)—water;
(iii)—1,2-propanediol;
(iv)—polyvinyl alcohol; and
(v)—an effective amount of at least one monomeric polyhydroxy compound:

2. The fixative composition according to claim 1, wherein the monomeric polyhydroxy compound is a carbohydrate comprising at least three carbon atoms.

3. The fixative composition according to claim 2, wherein the carbohydrate has six carbon atoms.

4. The fixative composition according to claim 3, wherein the carbohydrate is a hexitol.

5. The fixative composition according to claims 1, wherein the monomeric polyhydroxy compound is contained in an amount of from 0.05 to 2.00 wt.-%.

6. The fixative composition according to claim 5, wherein ethanol, water, 1,2-propanediol and polyvinyl alcohol are contained in an amount of from 50 to 85 wt.-%, 10 to 40 wt.-%, 3 to 20 wt.-% and 0.05 to 3.00 wt.-% respectively.

7. The fixative composition according to claim 6, wherein the ethanol concentration is 65 to 75 wt.-%.

8. The fixative composition according to claim 1, further containing additives and/or auxiliary agents.

9. The fixative composition according to claim 8, wherein the additives and/or auxiliary agents are selected from acceptable salts, alcohols, ketones, carboxylic acids, sugars and polymers.

10. A kit for fixation of tissue comprising a first means for keeping a mixture of water, 1,2-propanediol, polyvinyl alcohol and an effective amount of at least one monomeric polyhydroxy compound and a second means for keeping ethanol.

11. The kit according to claim 10, wherein the monomeric polyhydroxy compound is a carbohydrate comprising at least three carbon atoms.

12. The kit according to claim 11, wherein the carbohydrate has six carbon atoms.

13. The kit according to claim 12, wherein the carbohydrate is D-sorbitol.

14. The kit according to claim 10, wherein the contents of the first and the second means are mixed providing a fixative comprising 50 to 85 wt.-% of ethanol, 10 to 40 wt.-% of water, 3 to 20 wt.-% of 1,2-propanediol, 0.05 to 3.00 wt.-% of polyvinyl alcohol and 0.05 to 2.00 wt.-% of the monomeric polyhydroxy compound.

15. The kit according to claim 10, wherein the mixture of the first means further contains additives and/or auxiliary agents.

16. The kit according to claim 15, wherein the additives and/or auxiliary agents are selected from acceptable salts, alcohols, ketones, carboxylic acids, sugars and polymers.

17. A method for preparing a tissue sample for examination in histopathology, cytology or immunohistochemistry comprising the step of fixation of the tissue sample with a fixative composition comprising
(i)—ethanol;
(ii)—water;
(iii)—1,2-propanediol;
(iv)—polyvinyl alcohol; and
(v)—an effective amount of at least one monomeric polyhydroxy compound.

18. The method according to claim 17, wherein the tissue sample is of organs of human and/or animal origin.

19. The method of claim 18, wherein the tissue sample is a biopsy sample.

20. The method according to claim 17, wherein the tissue sample is fixed within a period of 15 minutes and 5 hours.

21. The method according to claim 19, wherein the biopsy sample is fixed within 20 minutes.

22. The method according to claim 17, wherein the tissue sample is a biopsy sample or surgical specimen and the method further comprises the step of testing the fixated tissue sample for proteins and/or nucleic acids.

23. The method according to claim 22, wherein the fixated sample or specimen is tested for an antigen.

24. The method according to claim 17, wherein the step of fixation is carried out in the presence of microwaves.
